(19) United States
(12) Patent Application Publication
(10) Pub. No.: US 2024/0415953 A1
(43) Pub. Date: Dec. 19, 2024

(54) ORAL THERAPEUTIC VACCINE COMPOSITIONS, METHODS AND TREATMENT OF COVID

(71) Applicant: Immunitor (Thailand) Co. Ltd., Chachoengsao (TH)

(72) Inventors: Vichai Jirathitikal, Chachoengsao (TH); Jonathan P. Wong, Burnaby (CA)

(73) Assignee: Immunitor (Thailand) Co. Ltd., Chachoengsao (TH)

(21) Appl. No.: 18/712,678

(22) PCT Filed: Nov. 21, 2022
(86) PCT No.: PCT/IB2022/061224

§ 371 Date: May 22, 2024

Related U.S. Application Data
(60) Provisional application No. 63/282,455, filed on Nov. 23, 2021.

(51) Int. Cl. A61K 39/215; A61K 9/00; A61K 9/20; A61K 39/00; A61P 31/14; A61P 37/06; C12N 7/00; C12N 15/113

(52) U.S. Cl. CPC A61K 39/215; A61K 9/0053; A61K 9/2009; A61K 9/2018; A61K 9/2059; A61P 31/14; A61P 37/06; C12N 7/00; C12N 15/1131; A61K 2039/542; C12N 2310/14; C12N 2770/20034

(57) ABSTRACT

Described herein are oral vaccine compositions for preventing and treating COVID and COVID related complications (e.g., cytokine storm related complications). These oral vaccine compositions comprise hydrolyzed and heat inactivated anti-viral antisense and other nucleic acid components that target the expression of SARS-CoV-2 viral proteins. Such oral vaccine compositions are room temperature stable and stimulate humoral (antibody), cellular and mucosal immunity.

### CROSS REFERENCE TO RELATED APPLICATIONS

This application claims the benefit of priority to U.S. Provisional Patent Application No. 63/282,455 filed Nov. 23, 2021, which is hereby incorporated by reference, in its entirety for any and all purposes.

### FIELD

The present technology is broadly related to molecular biology, virology, immunology, vaccinology. In particular, the present technology relates to oral compositions for the prevention and treatment of COVID and COVID related complications and that promote recovery from COVID-related organ damage.

### BACKGROUND

The current coronavirus disease 2019 (COVID-19) pandemic is devastating, causing heavy toll in human lives and severely hurting the global economy. As of Oct. 22, 2021, the World Health Organization (WHO) has reported more than 242 million confirmed cases of COVID-19 globally with more than 4.9 million deaths (www.who.int), thus making COVID-19 one of the world's deadliest pandemics in human history.

Most people exposed to COVID-19 will exhibit symptoms ranging from asymptomatic to mild and moderate illness. These people will naturally recover without requiring treatment or hospitalization. However, older people and those with underlying medical conditions, such as diabetes, cardiovascular diseases, obesity, and cancer, are more likely to develop severe illnesses. For instance, COVID-19 kills an estimated 13.4% of patients age 80 and older.

Currently, a number of COVID-19 vaccines had been approved by US FDA and WHO. These vaccines have so far been reported to be highly effective in protecting people from COVID, with efficacy rates ranging from 62-95%. Despite regulatory approval, vaccine hesitancy rates remain high as hundreds of thousand people on average are still succumbing to COVID globally each month. Furthermore, the emergence of new variants including the Delta variant further compromise the efficacy of current vaccines and undermine public confidence.

Current COVID vaccines are injectable formulations that are administered by health professionals. Such formulations are not room temperature stable and must be stored at low temperatures. Furthermore, current vaccines mainly target spike proteins, which are prone to random mutations and give rise to variants. Also, current vaccines stimulate humoral (antibody) immunity but not cellular and mucosal immunity. Significant and serious side effects of these vaccines include inflammation, allergy, and even death in rare cases. As such, there remains a need to develop COVID vaccines that address these limitations and disadvantages of current COVID vaccines.

With regard to COVID treatments, there is currently only one antiviral drug approved by the US FDA under emergency use authorization (EUA) to treat COVID-19 in adults and children who are age 12 and older. Remdesivir (Remdesivir, trade name: VEKLURY®, Gilead Sciences, US) is administered via injection into the vein. The use of remdesivir has been associated with elevated levels of liver enzymes (a sign for liver problems) and serious side effects including nausea and low blood pressure.

The treatment of COVID is further complicated by the ability of SARS-CoV-2 virus to cause a “cytokine storm” which is characterized by overreactive immune responses resulting in high levels of pro-inflammatory cytokines and chemokines. COVID patients with cytokine storm related complications often require hospital admission, suffer from acute respiratory distress syndrome (ARDS) needing oxygen ventilation, and have high fatality rate from multi-organ failure and blood clots. There are currently no existing drugs approved to treat COVID-related cytokine storm effects in COVID patients. Glucocorticoids, baricitinib, tocilizumab are anti-inflammatory drugs that can be targeted to calm the cytokine storm, and these have been shown to have some therapeutic outcomes in reducing mortality in COVID patients with severe cytokine storm related complications. Moreover, the impacts of COVID pandemic on mental health such as anxiety, depression and suicidal thoughts are profound, a recent study reported that more than a third of COVID patients suffered adverse neurological and psychiatric outcomes (Taquet et al., Lancet Psychiatry 2021, 8 (5): 416-427.)

Described herein are oral vaccine compositions for preventing and treating COVID and COVID related complications, e.g., cytokine storm related complications, that address the limitations associated with injectable vaccine compositions and current COVID drug treatments as described above. For instance, the oral vaccine compositions described herein are room temperature stable. Also, the oral vaccine composition described herein stimulate humoral (antibody) immunity with cellular and mucosal immunity, thus making these vaccines effective against COVID and its variants. In particular, these oral vaccine compositions described herein comprise hydrolyzed and heat inactivated anti-viral antisense and other nucleic acid components that target the expression of severe acute respiratory syndrome coronavirus 2 (SARS-CoV-2) viral proteins.

### SUMMARY

Provided in one aspect, is a composition comprising one or more hydrolyzed and heat inactivated anti-viral antisense and other nucleic acid components, wherein the composition is administered to a subject in need thereof.

In some embodiments, the one or more hydrolyzed and heat inactivated anti-viral antisense and other nucleic acid components comprise one or more genomic RNA sequences, immunomodulating CpG motifs, and small interfering RNAs (siRNAs). In some embodiments, the one or more hydrolyzed and heat inactivated anti-viral antisense and other nucleic acid components comprises one or more small interfering RNA (siRNA) targeting expression of SARS-CoV-2 viral proteins.

In some embodiments, the one or more hydrolyzed and heat inactivated anti-viral antisense and other nucleic acid components comprises one or more small interfering RNA (siRNA) comprising an oligonucleotide sequence as set forth in any one of SEQ ID NOS: 2, 4, 6, 8, 10, 12, 14, 16, 18, 20, 22, and 24. In some embodiments, the one or more hydrolyzed and heat inactivated anti-viral antisense and other nucleic acid components comprises one or more small interfering RNA (siRNA) comprising an oligonucleotide sequence as set forth in SEQ ID NO: 14, SEQ ID NO: 22, or SEQ ID NO: 24.

In any embodiments, the composition described herein further comprises one or more hydrolyzed and heat-inactivated SARS-associated coronavirus (SARS-CoV) antigens and alloantigens derived from pooled blood or serum of a species that is the same as the subject. In some embodiments, the one or more hydrolyzed and heat-inactivated SARS-CoV antigens and alloantigens comprise one or more selected from viral proteins, nucleocapsids, and genomic RNA sequences. In some embodiments, the one or more viral proteins comprise one or more glycoproteins, envelope and membrane proteins, nucleoproteins, and hemagglutinin-esterase proteins.

In any embodiments, the composition described herein further comprises one or more hydrolyzed and heat-inactivated immune-modulating agents. In some embodiments, the one or more one hydrolyzed and heat-inactivated immune-modulating agents having anti-inflammatory and antiviral activities. In some embodiments, the one or more one hydrolyzed and heat-inactivated immune-modulating agents comprise one or more immune-modulating cytokines, adjuvants, CpG motifs, peptides, RNA and DNA sequences, and anti-inflammatory molecules.

In any embodiments, the one or more hydrolyzed and heat inactivated anti-viral antisense and other nucleic acid components, one or more hydrolyzed and heat-inactivated SARS-associated coronavirus (SARS-CoV) antigens and alloantigens, one or more one hydrolyzed and heat-inactivated immune-modulating agents, or a combination thereof are each independently embedded in a matrix comprising one or more salts of a metal.

In some embodiments, the metal is selected from a group of alkali metals, alkaline earth metals, transition metals, metalloids, basic metals, and rare earth elements. In some embodiments, the metal is magnesium, calcium, lithium, beryllium, sodium, silicon, magnesium, aluminium, aluminum, potassium, titanium, vanadium, chromium, manganese, cobalt, nickel, copper, zinc, zirconium, molybdenum, silver, arsenic, cadmium, antimony, barium, osmium, platinum, or gold. In some embodiments, the metal is magnesium or calcium.

In some embodiments, the one or more salts of the metal comprise boride, acetate, phosphate, aluminide, aspartate, benzoate, bromide, carbonate, chloride, chloride hexahydrate, citrate, diboride, diglutamate, diuranate, fluoride, gluconate, hexahydrate, hydride, hydroxide, oxide, iodide, lactate, levulinate, nitrate, nitride, orotate, oxychloride, oxysulfate, perchlorate, peroxide, pidolate, silicide, stearate, sulfate, sulfide, sulfite, and trisilicate salt.

In some embodiments, the composition is formulated for oral administration, intranasal administration, intramuscular injection, aerosol delivery or intravenous injection. In some embodiments, the composition is formulated for oral administration. In some embodiments, the composition is formulated as a pill or tablet. In some embodiments, the composition further comprises one or more of excipients selected from a binder, starch or lactose, a disintegrating agent, a lubricant, a glidant, a sweetening agent, and a flavoring agent. In some embodiments, composition further comprises lactose monohydrate, tapioca starch, talc, and magnesium stearate.

In some embodiments, the composition is room temperature stable. In some embodiments, the composition is room temperature stable for at least one year, two years, three years, four years, or five years. In some embodiments, the composition does not require storage at low temperatures and/or refrigeration.

In some embodiments, the composition is administered to deliver a therapeutically effective amount of the one or more hydrolyzed and heat inactivated anti-viral antisense and other nucleic acid components to stimulate humoral (antibody), cellular, and mucosal immunity in the subject.

Provided in another aspect is a method of preventing or treating coronavirus disease 2019 (COVID-19) and COVID-19 related complications in a subject in need thereof comprising administering a therapeutically effective amount of any one of the compositions described herein. In some embodiments, the COVID-19 and COVID-related complications is caused by wildtype or variants of the SARS-CoV-2 virus. In some embodiments, the variant of the SARS-CoV-2 virus is the Delta variant.

Provided in another aspect is a method of treating hypercytokinemia (commonly called cytokine storm) and virus-induced organ damage associated with a virus selected from coronavirus disease 2019 (COVID-19), severe acute respiratory syndrome (SARS), Middle East respiratory syndrome (MERS), and influenza in a subject in need thereof comprising administering a therapeutically effective amount of any one of the compositions described herein. In some embodiments, the subject in need thereof is a human or an animal.

Provided in another aspect is a process of producing a solid oral composition as described herein, the process comprising:
(i) hydrolyzing one or more anti-viral antisense and other nucleic acid components;
(ii) precipitating the one or more anti-viral antisense and other nucleic acid components with one or more salts of a metal;
(iii) heat treating the one or more anti-viral antisense and other nucleic acid components to provide one or more hydrolyzed and heat inactivated anti-viral antisense and other nucleic acid components; and
(iv) formulating one or more hydrolyzed and heat inactivated anti-viral antisense and other nucleic acid components into the solid oral composition.

Provided in another aspect is a process of producing a solid oral composition as described herein, the process comprising:
(i) hydrolyzing one or more anti-viral antisense and other nucleic acid components and one or more SARS-CoV antigens and alloantigens;
(ii) precipitating one or more anti-viral antisense and other nucleic acid components and one or more SARS-CoV antigens and alloantigens with one or more salts of a metal;
(iii) heat treating one or more anti-viral antisense and other nucleic acid components and one or more SARS-CoV antigens and alloantigens to provide one or more hydrolyzed and heat inactivated anti-viral antisense and other nucleic acid components and one or more one or more hydrolyzed and heat inactivated SARS-CoV antigens and alloantigens; and
(iv) formulating one or more hydrolyzed and heat inactivated anti-viral antisense and other nucleic acid components and one or more hydrolyzed and heat inactivated SARS-CoV antigens and alloantigens into the solid oral composition.

In some embodiments, hydrolyzing comprises acid treatment. In some embodiments, precipitation provides for the formation a metal salt matrix, wherein the one or more anti-viral antisense and other nucleic acid components and optionally, the one or more SARS-associated coronavirus (SARS-CoV) antigens and alloantigens, are embedded in the metal salt matrix.

### BRIEF DESCRIPTION OF THE DRAWINGS

FIGS. 1A, 1B, and 1C illustrate the mechanism of action of HAP-V+ against SARS-CoV-V2 virus. FIG. 1A shows the 3 groups of active biologicals (antisense, viral antigens and immunomodulating agents that are embedded in Magnesium matrix nanopartciles. These particles protect these agents from acid digestion and from enzymatic breakdown in the gastrointestinal tract. FIG. 1B shows the actions of antisense and/or siRNA that block virus entry, inhibit the viral gene expression and blocking of the viral mRNA expression. FIG. 1C shows inhibition of viral gene expression of viral polymerase and envelope proteins by ASO and siRNA as indicated by PCR reactions. HAP-V+ and HAP-VL contain antisense components embedded in magnesium matrix that binds to the SARS-CoV-2 virus, neutralizes virus and blocks virus entry into host cells, and stimulates the mucosal immune responses to provide broad and systemic antiviral response that protect hosts from infection to spreading in the body.

FIG. 2 illustrates the Insert Design for pSilencer 1.0-U6. The RNA oligonucleotides consisted of a 19-nucleotide sense siRNA sequence linked to its reverse complementary antisense siRNA sequence by a short spacer. This step had been successfully achieved by using a 9-nucleotide spacer (TTCAAGAGA), although other spacers can be designed. 5-6 T's were added to the 3′ end of the oligonucleotide. In addition, for cloning into the pSilencer 1.0-U6 vector, nucleotide overhangs to the EcoR I and Apa I restriction sites were added to the 5′ and 3′ end of the DNA oligonucleotides, respectively.

FIG. 3 illustrates the Insert Design for pSilencer 2.0-U6 and pSilencer 3.0-H1. The insert design is specific for the pSilencer 2.0-U6, 2.1-U6, 3.0-H1 and 3.1-H1 Expression Vectors and contains the appropriate overhanging 5′ ends for directional cloning into these plasmids. As with p Silencer 1.0-U6 shown in FIG. 2, early indications suggest that a great deal of latitude is possible in the design of the loop.

FIGS. 4A, 4B, and 4C show the inhibition of cytopathogenic effects (CPE) and infectivity of SARS-CoV-2 wildtype strain in African green monkey's kidney epithelial cells (Vero cells). FIG. 4A shows the neutralization of SARS-CoV-2 virus by various concentrations of HAP-V+ (1-2 mg/mL) in Vero cells as measured by the cytopathathic effect (CPE) and cycle threshold (CT) at day 1-3 post treatment. FIG. 4B shows the inhibition of viral replication by HAP-V+ in Vero cells by measured by the number of viral RNA copy numbers at day 1-3 post treatment. FIG. 4C shows neutralization of SARS-COV-2 by HAP-V+ as expressed as % viral inhibition.

FIGS. 5A and 5B show the inhibition of cytopathogenic effects (CPE) and infectivity of SARS-CoV-2 Delta variant strain in African green monkey's kidney epithelial cells (Vero cells). FIG. 5A shows the neutralization of Delta variant strain of SARS-CoV-2 virus by various concentrations of HAP-V+ (0.25 to 20 mg/mL) in Vero cells as measured by the cytopathathic effect (CPE) and cycle threshold (CT) at day 2-5 post treatment FIG. 5B shows inhibition of viral infectivity of the Delta variant strain of SARS-CoV-2 as measured using the CPE assay and expressed as cycle threshold (CT) reduction values.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, administration of the vaccines to a subject in need thereof who is a non-infected host is referred to as “preventive or prophylactic vaccination” while administration to a subject in need thereof who is a host already infected is referred as “therapeutic vaccination.” The term “prevention” includes an immunological intervention to protect the subject in need thereof or the host from occurrence of a disease or condition before it happens. The term “treatment” includes effort to alleviate the disease or clinical conditions of a subject in need thereof.

As used herein, an alloantigen is an antigen presented in allelic forms as encoded at the same gene locus in different individuals of the same species. Alloantigen can be any protein, peptide, amino acid, or nucleic acid that serves as an alloantigen.

As used herein, the term “adjuvant” refers to any compound which, when injected together with an antigen, non-specifically enhances the immune response to that antigen. While the preferred mode of instant administration is without and adjuvant, one skilled in the art, can administer the composition with an immune adjuvant known in the art. Of particular advantage are those that favorably induce immune tolerance and yet at same time produce beneficial clinical effect and capable of clearing the infection.

As used herein, a “patient” refers to a host, such as a human capable of being infected with the COVID-19 virus. A patient may or may not be infected with COVD-19 virus. Examples of patients are humans, chimpanzees, ducks, pigs, dogs, cats, woodchucks and other species capable of being infected with COVID-19.

As used herein, the term “antigen” means a molecule which contains one or more epitopes capable of stimulating a host's immune system to make a cellular immune response when the antigen is presented in accordance with the present invention, or a humoral antibody response. The capacity to induce the immune response is understood as “immunogenicity.”

As used herein, the “therapeutically effective amount” implies an amount that will be included in the composition which will cause the subject to produce a sufficient response, in order to prevent, reduce, eliminate or diagnose symptoms. The exact amount necessary will vary, depending on the subject being treated; the age and general condition of the subject to be treated; the severity of the condition being treated; in the case of an immunological response, the capacity of the subject's immune system to synthesize antibodies; the degree of protection desired and the particular antigen selected and its mode of administration, among other factors. An appropriate effective amount can be readily determined by one of skill in the art. Thus, a “therapeutically effective amount will fall in a relatively broad range that can be determined through routine experimentation such as a clinical trial.

The current COVID pandemic has exposed many inherent shortfalls in the prevention and treatment of pandemic viruses. Injectable vaccines can take long time to develop and they can elicit many side effects that result in high vaccine hesitancy rate. Furthermore, these injectable vaccines require strict refrigeration or freezing conditions for stability and their efficacy is compromised by emergence of mutant variants. Furthermore, while these major COVID vaccines are costly to research and develop, these vaccines do not offer therapeutic benefits to people already infected with COVID and are exhibiting COVID symptoms.

Described herein are oral vaccine compositions that address the limitations and disadvantages of current injectable vaccines. The oral vaccine compositions described herein are useful for preventing and treating COVID and COVID related complications (e.g., cytokine storm related complications). These oral vaccine compositions comprise hydrolyzed and heat inactivated anti-viral antisense and other nucleic acid components that target the expression of severe acute respiratory syndrome coronavirus 2 (SARS-CoV-2) viral proteins. The oral vaccine compositions described herein are also room temperature stable and have improved stability, including storage stability, over injectable vaccine formulations.

There is an urgent need for COVID vaccines and antiviral drugs that are effective against both wildtype and emerging variants of SARS-CoV-2. Most of the current COVID-vaccines are designed to target the spike protein, which is prone to random mutations and gives rise to variants. As such, vaccines that provide neutralizing antibodies against the spike protein may therefore be ineffective. To address this, the oral vaccine compositions described herein are designed to target multiple sites of virus replication, and induce both humoral (antibody), cellular (lymphocytes) and mucosal immunity. Mucosal immunity is often overlooked but it is fundamentally important because SARS-CoV-2 is a mucosal pathogen that enters and infect primarily on mucosal surfaces of the body. Thus, the oral vaccine compositions described herein are room temperature stable and stimulate humoral (antibody) immunity with cellular and mucosal immunity.

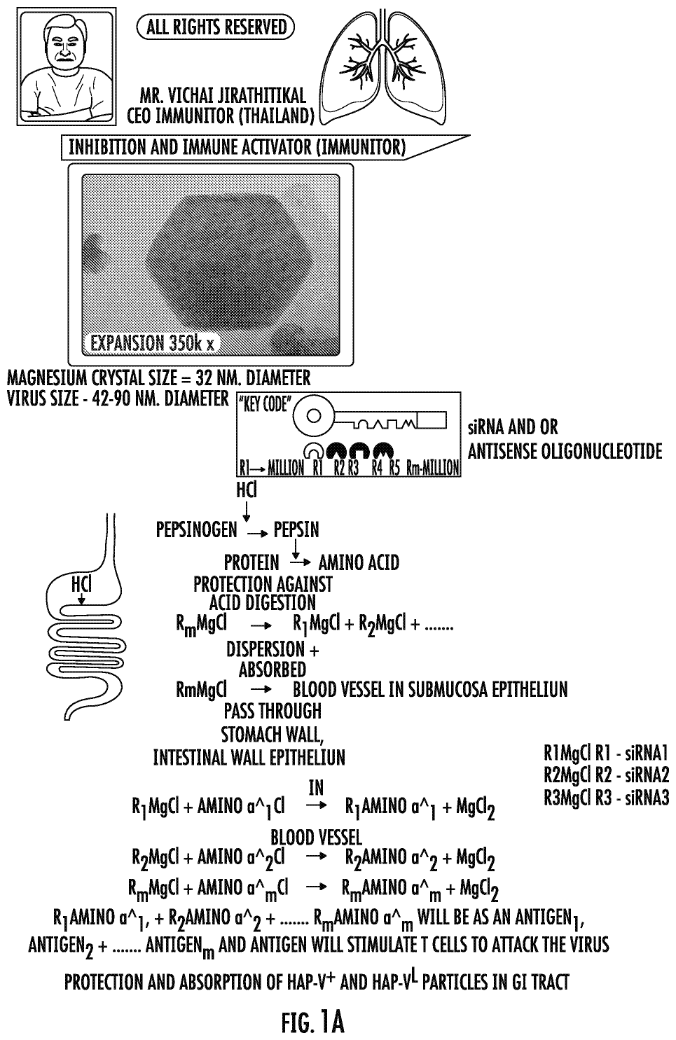

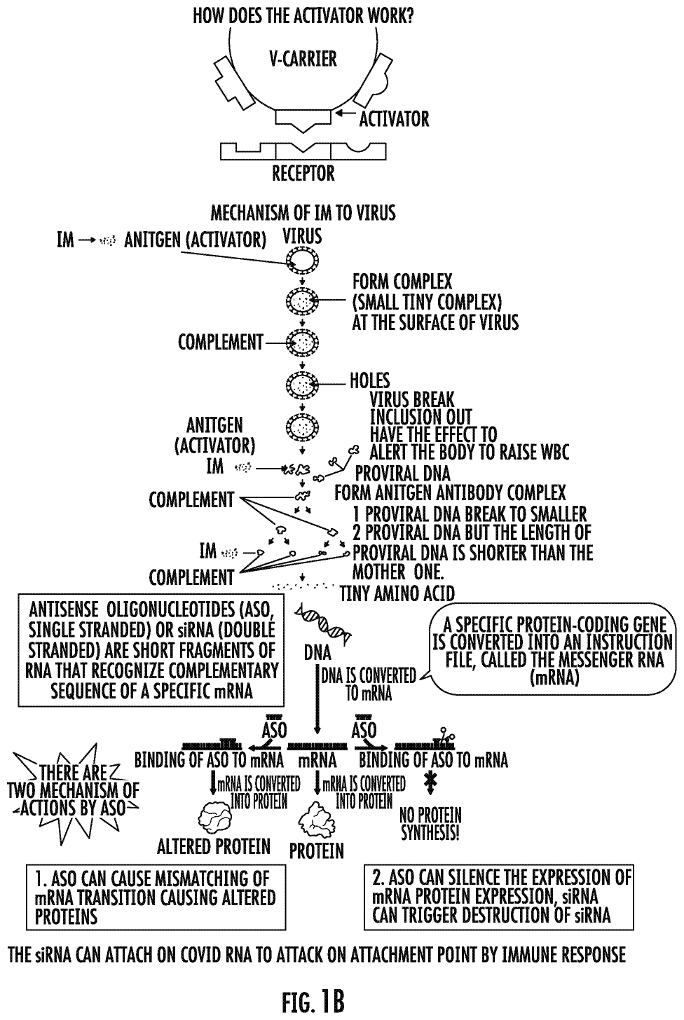

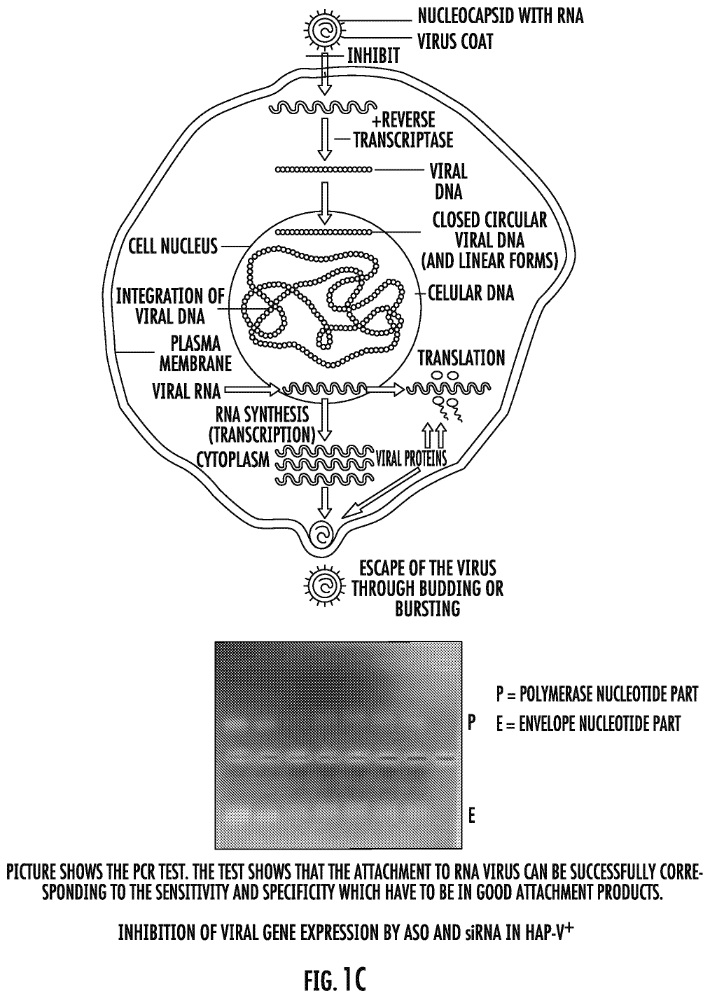

The vaccine compositions described herein (e.g., HAP-V+ and HAP-VL) are examples of an intersecting platform that integrates Immunitor's oral therapeutic vaccine technology with antisense molecules embedded in nanoparticle matrix as delivery system. The feasibility of such intersecting technologies is currently being explored for potential applications in the prevention and treatment of infectious and malignant diseases. The primary mechanism for the antiviral effect of HAP-V+ is mediated through a “key lock” mechanism, which prevents the virus from entering into host target cells. While HAP-VL works by treating cytokine storm-associated lung and organ damage caused by SARS-CoV-2. Both HAP-V+ and HAP-VL are embedded in a metal salt matrix (e.g., a magnesium salt matrix) that complexes with inactivated SARS-CoV-2 viral antigens. These complexes act as effective locks that block entry and neutralize the infectivity of the SARS-CoV-2 virus (FIGS. 1A, 1B, and 1C). FIG. 1A shows the 3 groups of active biologicals (antisense, viral antigens and immunomodulating agents that are embedded in Magnesium matrix nanopartciles. These particles protect these agents from acid digestion and from enzymatic breakdown in the gastrointestinal tract. FIG. 1B shows the actions of antisense and/or siRNA that block virus entry, inhibit the viral gene expression and blocking of the viral mRNA expression. FIG. 1C shows inhibition of viral gene expression of viral polymerase and envelope proteins by ASO and siRNA as indicated by PCR reactions. Because HAP-V+ is administered orally and gastrointestinal surfaces are densely populated with mucosal immune cells, the COVID-19 virus antigens are protected by the metal salt matrix (e.g., magnesium matrix) from gastric digestion in the stomach and thus can elicit safe and effective antiviral immune responses with humoral, cellular, and mucosal involvement.

The HAP-V+ and HAP-VL compositions described herein have similar components; however, the HAP-VL compositions further comprise one or more immune-modulating agents. These immune-modulating agents include anti-inflammatory and/or lung and brain tissue healing molecules that are not found in the HAP-V+ formulation and are further described herein.

As shown in the preclinical studies described in the Examples, HAP-V+ has been shown to demonstrate a high degree of neutralization of SARS-CoV-2 infectivity and inhibition of viral replication in mammalian cell culture system although HAP-V+ and HAP-VL have not been tested in human clinical trials for safety and efficacy against COVID. Furthermore, magnesium (Mg2+) has been shown to play a vital role in regulating many cellular, metabolic, and immune functions. Magnesium deficiency has been associated with increased susceptibility to infectious and neoplastic diseases. More importantly, people with low blood magnesium levels may have an increased susceptibility to be infected with COVID-19 and are likely to face severe disease progression from early symptoms to severe clinical outcomes, including death.

The oral vaccine compositions described herein are thermal stable and do not require refrigeration. Furthermore, the oral vaccine compositions may be formulated as a pill and does not require administration by a health professional as required for injectable vaccines. Also, the oral vaccines described herein target multiple sites and not just spike proteins. These oral vaccines are also safe and do not cause significant side effects. Also, as the oral vaccines described herein target and stimulate mucosal immunity, these oral vaccines have the potential of being effective against new variants of COVID-19.

### Anti-Viral Antisense Components

The compositions described herein may comprise one or more anti-viral antisense and other nucleic acid components, wherein one or more anti-viral antisense components and other nucleic acid are hydrolyzed and heat inactivated. In some embodiments, the one or more hydrolyzed and heat inactivated anti-viral antisense and other nucleic acid components comprise one or more genomic RNA sequences, immunomodulating CpG motifs, and small interfering RNAs (siRNAs). In some embodiments, the one or more hydrolyzed and heat inactivated anti-viral antisense and other nucleic acid components comprises one or more small interfering RNA (siRNA) targeting expression of SARS-CoV-2 viral proteins.

In some embodiments, the one or more hydrolyzed and heat inactivated anti-viral antisense and other nucleic acid components comprises one or more small interfering RNA (siRNA) comprising an oligonucleotide sequence as set forth in any one of SEQ ID NOS: 2, 4, 6, 8, 10, 12, 14, 16, 18, 20, 22, and 24.

In some embodiments, the one or more hydrolyzed and heat inactivated anti-viral antisense and other nucleic acid components comprises one or more small interfering RNA (siRNA) comprising an oligonucleotide sequence as set forth in SEQ ID NO: 14, SEQ ID NO: 22, or SEQ ID NO: 24.

### Alloantigens

The compositions described herein may further comprise one or more SARS-associated coronavirus (SARS-CoV) antigens and alloantigens, wherein these SARS-associated coronavirus (SARS-CoV) antigens and alloantigens are hydrolyzed and heat-inactivated.

Viral antigens, such as the SARS-associated coronavirus (SARS-CoV) antigens, are viral components, which include but are not limited to viral proteins, RNA or DNA genomic sequences, and nucleocapsids. Alloantigens are antigens associated with self vs non-self recognition (e.g blood type or antigens). Alloantigen can be any protein, peptide, amino acid, or nucleic acid that serves as an alloantigen. In some embodiments, the SARS-associated coronavirus (SARS-CoV) antigens and alloantigens are derived from pooled blood or serum of a species that is the same as the subject.

In some embodiments, the one or more hydrolyzed and heat-inactivated SARS-associated coronavirus (SARS-CoV) antigens and alloantigens comprise one or more selected from viral proteins, nucleocapsids, and genomic RNA sequences. In some embodiments, the one or more viral proteins comprise one or more glycoproteins, envelope and membrane proteins, nucleoproteins, and hemagglutinin-esterase proteins.

The source of viral antigens does not necessarily need to be from blood or blood derivatives such as plasma or serum. Sputum or other body fluids like urine, gastrointestinal secretions, liquid of tissues or tumors, synovial fluid, saliva, sputum, cyst fluid, amniotic fluid, cerebrospinal fluid, peritoneal fluid, lung lavage fluid, semen, lymphatic fluid, tears, perspirations, and prostatic fluid may be used as a source of the viral antigens. These fluids may contain leukocytes, macrophages or other cells that are equally advantageous. Without limiting to fluids, solid tissues such as lung, liver, pancreas, skeletal muscle, heart, kidney, brain, bone marrow, tumor tissue, skin, myelin, collagen, feces, and other tissues may also be used.

Whole blood, or serum or plasma, or culture broth with infectious whole virions present in them are treated preferably with an acid or alkali. Cell lines like Vero and MDCK cells, for example, can be used equally to propagate SARS-CoV-2 and influenza viruses. One skilled in the art may use other hydrolyzing agents like enzymes or detergents. Samples to be hydrolyzed are pooled or from one individual, or maintained as a separate batch.

In any case the amount of SARS-CoV antigen or alloantigen shall not be too low so that it could be considered as a contaminant. For compositions made from pooled plasma, the amount of SARS-CoV antigen or alloantigen varies and depends on the batch or the manufacturer; however, the alloantigen is usually present in amount of less than 1%. In the case of the hepatitis A vaccine, the WHO recommends the amount of contaminating albumin to be less than 50 nanograms per dose. Thus, these requirements distinguish the instant composition from classical vaccine compositions of prior art, since in the present invention the ratio of various SARS-CoV antigen or alloantigen molecules to antigen is always higher than 1:1, preferably more 10:1, ideally more than 100:1 and always exceeds 50 nanogram/gram when albumin is used to gauge the amount of allogeneic material. As intended herein, more than one type alloantigen is contemplated. Such compositions, especially oral dosage forms, will be useful to a subject in need thereof, such as a human host or other species in need thereof.

### Immune-Modulating Agents

The compositions described herein may further comprise one or more hydrolyzed and heat-inactivated immune-modulating agents. In some embodiments, the hydrolyzed and heat-inactivated immune-modulating agents having anti-inflammatory and antiviral activities.

Illustrative examples of immuno-modulating agents include, but are not limited, to synthetic and natural molecules that modify the host's immune system, and these include immunomodulating cytokines, adjuvants, CpG motifs, peptides, RNA and DNA sequences. In some embodiments, the one or more one hydrolyzed and heat-inactivated immune-modulating agents comprise one or more immune-modulating cytokines, adjuvants, CpG motifs, peptides, RNA and DNA sequences, and anti-inflammatory molecules.

### Metal Salts

As described herein, the compositions are effective in their dosage form (e.g., the oral dosage form) as the components (e.g., an antigen, an antisense, and immune-modulating agents) are embedded or entrapped within a pharmaceutically acceptable metal salt matrix. Embedment and entrapment of the composited components protects these components from gastric digestion in the gastrointestinal tract, enables these components to retain their biological and immunological properties

The difference between present composition and prior art compositions having an antigen mainly adsorbed to the surface of an immune adjuvant, such as aluminum hydroxide, is that the instant antigen is embedded within a matrix, which keeps the physical structure intact even at conditions that are commonly unfavorable, i.e., high temperature with long periods of time. As such, the compositions described herein do not require using an immune adjuvant to produce the desired effect when the composition is administered orally. In some embodiments, the composition described herein are administered without an immune adjuvant. While this is the preferred mode of administration, one skilled in the art can administer the composition with an immune adjuvant known in the art.

In some embodiments, wherein the one or more hydrolyzed and heat inactivated anti-viral antisense and other nucleic acid components, one or more hydrolyzed and heat-inactivated SARS-associated coronavirus (SARS-CoV) antigens and alloantigens, one or more one hydrolyzed and heat-inactivated immune-modulating agents, or a combination thereof are each independently embedded in a matrix comprising one or more salts of a metal.

The hydrolyzed antigens (including viral antigens) and alloantigens described herein may be embedded in metal salts. In some embodiments, the metal is selected from metal is selected from a group of alkali metals, alkaline earth metals, transition metals, metalloids, basic metals, and rare earth elements. In some embodiments, the metal is magnesium, calcium, lithium, beryllium, sodium, silicon, magnesium, aluminium, aluminum, potassium, titanium, vanadium, chromium, manganese, cobalt, nickel, copper, zinc, zirconium, molybdenum, silver, arsenic, cadmium, antimony, barium, osmium, platinum, or gold. In some embodiments, the metal is magnesium or calcium.

The salts of the metal used herein include, but are not limited to boride, acetate, phosphate, aluminide, aspartate, benzoate, bromide, carbonate, chloride, chloride hexahydrate, citrate, diboride, diglutamate, diuranate, fluoride, gluconate, hexahydrate, hydride, hydroxide, oxide, iodide, lactate, levulinate, nitrate, nitride, orotate, oxychloride, oxysulfate, perchlorate, peroxide, pidolate, silicide, stearate, sulfate, sulfide, sulfite, and trisilicate salt.

Representative magnesium salts include but are not limited to, aluminum magnesium boride, calcium magnesium acetate, dimagnesium phosphate, magaldrate, magnesium aluminide, magnesium aspartate, magnesium benzoate, magnesium bromide, magnesium carbonate, magnesium chloride, magnesium chloride hexahydrate, magnesium citrate, magnesium diboride, magnesium diglutamate, magnesium diuranate, magnesium fluoride, magnesium gluconate, magnesium hexahydrate, magnesium hydride, magnesium hydroxide, magnesium iodide, magnesium lactate, magnesium levulinate, magnesium nitrate, magnesium nitrite, magnesium orotate, magnesium oxide, magnesium oxychloride, magnesium oxysulfate, magnesium perchlorate, magnesium peroxide, magnesium phosphate, magnesium pidolate, magnesium silicide, magnesium stearate, magnesium sulfate, magnesium sulfide, magnesium sulfite, magnesium trisilicate, monomagnesium phosphate, tri-magnesium citrate and the like.

In a further aspect, two or more metals may be used simultaneously. For example, calcium chloride and magnesium chloride can be mixed at various acceptable ratios into which hydrolyzed antigens are embedded.

### Formulations

The compositions described herein comprise antisense molecules, other nucleic acid components, immune-modulating agents, antigens from pathogen-infected and non-infected tissues derived from the same species as the host itself (allogeneic component or alloantigen) that may be formulated into a pill or tablet having carrier molecules of metal salts. The compositions described herein overcome the challenges inherent in many conventional delivery systems and serve to introduce a safe and non-toxic modifying agent, a prophylactic and therapeutic vaccine, into and across the mucosal membrane of a human or animal subject. The compositions may be administered through a mucosal surface, e.g., enterally by an oral route, to provide significant clinical benefit to subjects in need thereof. The delivery of such vaccine components by the carrier delivery system enables the vaccine components to be delivered directly into the primary site of infection for SARS-CoV-2, SARS-CoV-1, MERS, influenza viruses, i.e., mucosal surfaces, which are interconnected in the entire body, and thus providing a systemic protective antiviral responses which the viral entry and replication in these sites.

While the preferred dosage form of the compositions described herein is an oral solid dosage form, such as a tablet or pill, the composition described herein may be formulated into any suitable dosage form, including but not limited to, aqueous oral dispersions, aqueous oral suspensions, aerosols, controlled release formulations, fast melt formulations, effervescent formulations, self-emulsifying dispersions, solid solutions, liposomal dispersions, lyophilized formulations, capsules, powders, delayed release formulations, immediate release formulations, modified release formulations, extended release formulations, pulsatile release formulations, multiparticulate formulations, and mixed immediate release formulations.

The oral compositions described herein may further comprise one or more excipients. A suitable excipient includes an inert substance which is added to an active ingredient to provide bulk, for example in tablets. Illustrative components include the following ingredients, or compounds of a similar nature: a binder, such as microcrystalline cellulose, gum, or gelatin; an excipient, such as starch or lactose; a disintegrating agent, such as alginic acid or corn starch; a lubricant, such as magnesium stearate; a glidant, such as colloidal silicon dioxide; a sweetening agent, such as sucrose or saccharin; and a flavoring agent, such as peppermint or some other flavoring. The excipients may be incorporated into the compositions described herein by a wet or dry granulation processes well known to formulation experts and to provide a resulting mixture that may then be further compressed into tablets.

In certain embodiments, the composition further comprises one or more of excipients selected from a binder, starch or lactose, a disintegrating agent, a lubricant, a glidant, a sweetening agent, and a flavoring agent. In specific embodiments, the composition further comprises lactose monohydrate, tapioca starch, talc, and magnesium stearate.

The properties of the excipients used herein that may have an influence on the final composition, such as moisture content, particle size and distribution, polymorphism, amorphism, crystal habit, hydration state, lubrication, and binder level of the blend, are within routine knowledge of a practitioner.

The mechanistic aspects of tableting, such as choice of punches/dies, tableting machines, can be selected without undue experimentation by those skilled in the art. In addition, the compositions described herein may further include various other materials that may modify the physical form, for example, coatings of sugar, shellac, or other enteric agents. The tablets described herein may be covered with an enteric coating material that is predominantly soluble in the intestinal fluid, but substantially insoluble in the gastric fluids of stomach. Preferred enteric coating materials are chosen from those that are commercially available, although new materials can be selected that are with the skill of practicing artisan.

### Frequency of Administration

The compositions described herein may be administered for one month to one year, or carried out for as many years as deemed necessary. Treatment can be stopped after 1-3 months of treatment. Treatment may be repeated again after one month of break, although in some patients repeat treatment is not necessary for as long as 3 or 6 months. In some instances, it is preferred to stop for one year and then repeat again. In some individuals, treatment may not to be repeated at all as patients are cured and need no further dosing.

In some embodiments, compositions described herein provide a therapeutically effective amount of composition over an interval of about 3 hours to about 24 hours after administration, enabling, for example, once-a-day, twice-a-day (b.i.d.), or three times a day (t.i.d.) administration if desired. A simple dosing regimen of one or two tablets per day is preferred. Administration can be a single tablet administration, more preferably repeated administration for at least one week up.

### Active Agent

The amount of active ingredient in the compositions described herein (e.g., tablets or pill) include the active agent (e.g., anti-viral antisense and other nucleic acid components, SARS-CoV antigens and alloantigens, immune-modulating agents, or a combination thereof), that is administered to a subject via a solid dosage form. In some embodiments, the amount of the active agent present in the solid dosage form, such as a tablet or pill, is the amount known in the art to achieve a therapeutically effective concentration in a human or an animal in need thereof.

For example, the amount of one or more active agents may range from about 0.0001 micrograms to about 1 g. In other embodiments, the amount of active agents may range from about 0.001 micrograms to about 100 mg. In other embodiments, the amount of active agents may preferably range from about 0.01 microgram to about 10,000 microgram, from about 0.5 microgram to about 1,000 microgram or from about 1 microgram to about 500 microgram. In another embodiment, a formulation is administered in a solid dosage form at a total protein concentration of about 10 microgram to about 500 microgram. In another embodiment, the active agents in the formulation is administered in a solid dosage form at concentration of about 1-100 microgram. In other embodiment the amount of active agents is from about 0.00001% to about 30% based on the total weight of the composition, preferably the amount is between about 0.001% and 20%, preferably the amount is between about 0.01% and 10%, preferably the amount is between about 0.1% and 5%.

The amount of the active agents can also vary depending on the intended use. For therapeutic use, the amount can be higher by about one order of magnitude from the amount of the composition intended for prophylactic use. For example, if in a therapeutic composition the quantity of active ingredient is 60 microgram, then in a prophylactic version the dose can be reduced to 5 or 10 micrograms. However, this is not an absolute requirement. These and other implications related to effective dose are not intended to be limiting and are provided by a way of example only. The precise amount of active agents that is “therapeutically effective” is determined by standard clinical testing whereby the clinical effect is monitored by means well known to those skilled in the art.

### Stability

As described herein, the compositions described here are stable (e.g. at room temperature stable) and as such, also have long shelf lives of more than 1 year, 2 years, 3 years, 4 years, or 5 years. In some embodiments, the compositions are oral tablets that are thermal-stable, do not require refrigeration/freezing, and have long shelf lives of more than 3 years.

In some embodiments, the composition is room temperature stable. In some embodiments, the composition is room temperature stable for at least one year, two years, three years, four years, or five years. In some embodiments, the composition does not require storage at low temperatures and/or refrigeration.

### Methods of Making

The compositions described herein are prepared by a 4-step manufacturing and production technology platform that enables the compositions to be mass produced without long preparative steps and times. Since the preferred compositions are oral tablets, then the compositions described here are thermal-stable, do not require refrigeration/freezing, and have long shelf lives of more than 3 years. Furthermore, they can be side administered orally and therefore do not require injections with the assistance support from health professionals, and are free of the many side effects with injectable vaccines, which may include pain and muscle ache, fever, malaise, inflammation and allergic reactions.

The key objective of the 4-step production process used to prepare the compositions described herein is to inactivate any infectious or toxic component of the composition by acid-hydrolysis and heat activation by autoclaving while retaining the biological and immunological properties of each component. As discussed herein, the biological and immunological properties of each component is maintained by embedding these components (e.g., anti-viral antisense and other nucleic acid components, SARS-associated coronavirus (SARS-CoV) antigens and alloantigens, immune-modulating agents, or a combination thereof)) in the metal salt carrier matrix (e.g., magnesium, calcium and other inorganic salts) thus protecting these components from inactivation. These compositions can then be formulated into an powder that may be then further compacted into an oral pill/tablet as the final processed product.

Hydrolysis is one steps of the 4-step process used to prepare the compositions described herein. Hydrolysis can be achieved by acid treatment as the preferred mode of hydrolysis. The pH range is preferably below 6 and more preferably between 0.5 and 5.5, more preferably between 1 and 4, more preferably between 1.0 and 3.0, and even more preferably between 1.0 and 2.0. While these ranges are given as optimal ranges, they will not prevent one skilled in the art to determine additional ranges, which remain within the scope of the present invention.

The proportion of hydrolyzed antigen (e.g., anti-viral antisense and other nucleic acid components, SARS-associated coronavirus (SARS-CoV) antigens and alloantigens, immune-modulating agents, or a combination thereof), is at least 10% by weight of the 50 non-hydrolyzed native antigen, preferably at least 20%, preferably at least 30%, preferably at least 40%, preferably at least 50%, preferably at least 60%, preferably at least 70%, preferably at least 80%, and can be at least 90%. The meaning of 100% hydrolyzation is a situation when all proteins are hydrolyzed to free amino acids without any larger residue, such as a peptide, remaining after the hydrolysis reaction.

The duration of the hydrolysis can be anywhere between 5 minutes to several hours, more preferably they are between 60 10 minutes and 5 hours, even more preferably between 20 minutes and 3 hours. The duration can be a shorter period of hours, which is determined by simple experimentation under routine hydrolysis conditions well familiar to those skilled in the art. Thus, the duration of hydrolysis can be 30 minutes, 65 one hour, 1.5 hours, 2 hours, 2.5 hours, or 3 hours, or intermediate times.

The acids that are useful for hydrolysis can be, but are not limited to, inorganic acids, such as hydrochloric acid, hydrobromic acid, sulfuric acid, nitric acid, carbonic acid, phosphoric acid and the like. Examples of organic acids include, but are not limited to, for example, acetic acid, citric acid, propionic acid, glycolic acid, pyruvic acid, oxalic acid, malic acid, malonic acid, succinic acid, maleic acid, fumaric acid, tartaric acid, benzoic acid, cinnamic acid, mandelic acid, methanesulfonic acid, ethanesulfonic acid, salicylic acid, p-toluenesulfonic acid, and the like.

In one embodiment when the composition is still in liquid solution and in process of hydrolysis, the products of hydrolysis can be precipitated with a salt of a metal or with an alkaline metal hydroxide solution that forms a salt. This step effectively ends the hydrolysis since the components (e.g., anti-viral antisense and other nucleic acid components, SARS-associated coronavirus (SARS-CoV) antigens and alloantigens, immune-modulating agents, or a combination thereof) are entrapped or embedded within the matrix of the solid matter and do not react with the hydrolytic agent. The precipitation can be run simultaneously with hydrolysis or started after the hydrolysis step, depending on the intended use of the final product. For example, an aqueous solution comprising the anti-viral antisense and other nucleic acid components, SARS-associated coronavirus (SARS-CoV) antigens and alloantigens, immune-modulating agents, or a combination thereof, preferably about 90% to about 20% protein, and more preferably about 80% to about 40% protein solution, is treated with an acid, until the pH is about 1 to about 6, preferably about 1 to about 4, more preferably about 1 to about 2. The solution is stirred at low speed, preferably at about 10 to about 500 rpm, more preferably about 30 to about 300, even more preferably about 60 to about 200 rpm for about 60 minutes to about 120 minutes, preferably about 45 to about 80 minutes, more preferably about 30 to about 60 minutes. A metal salt is then added to the stirring solution to create a slurry that solidifies as a solid mass. The mass is washed for about 1 minute to about 60 minutes, preferably about 5 to about 45 minutes, more preferably about 10 to about 30 minutes and then dried in an oven by heating at about 56 to about 200° C., preferably at about 70 to about 150° C., more preferably about 80 to about 140° C., more preferably about 100 to about 120° C., for about 1 hour to about 72 hours, preferably about 3 to about 48 hours, more preferably about 5 to about 12 hours. The dried solid mass is then reduced to a powder.

It is equally advantageous to start the precipitation and add hydrolyzant after that step. In yet another embodiment the metal salt is present prior to the hydrolysis and proteins are hydrolyzed in the presence of said metal salts, to be precipitated simultaneously and concomitantly. Representative steps for process of embedding an active agent (e.g., anti-viral antisense and other nucleic acid components, SARS-CoV antigens and alloantigens, immune-modulating agents, or a combination thereof) can include contacting the active agent with a metal salt solution to create a slurry and hydrolyzing the resulting 5 mixture by contacting it with an acid so that this mixture is hardened and transforms into a solid phase or a solid state form-a process also know as a setting of the solid matter. This solid form is then reduced to powder particles embedded within active agent. The sequence of these steps are easily determined beforehand and can be carried out without undue experimentation as long as the final product with active agent is clinically effective as determined by clinically effective as determined by clinical studies.

The next step in the this process is thermal treatment, which inactivates the active ingredients (e.g., anti-viral antisense and other nucleic acid components, SARS-CoV antigens and alloantigens, immune-modulating agents, or a combination thereof). It is well known that exposure to high temperature, for example, heating at 56° C. or higher, like 80° C. for one hour, destroys adventitious pathogens but not useful for making vaccines. However, the compositions described herein may be exposed to high temperatures without losing antigenicity and exhibit a positive clinical benefit. Thus, as the compositions are heat treated (exposed to high temperatures for one hour or more), any potentially infectious viruses that may be present within the composition is effectively killed, thus providing a final composition that is safe to administer to subjects in need thereof.

The resulting compositions after heat treatment are then further processed as described above to provide a final dosage form that may be administered to a subject in need thereof (e.g., a solid oral dosage form, such as a tablet or pill).

Provided in one aspect is a process of producing a solid oral composition according to any one of the compositions described herein, the process comprising:
(i) hydrolyzing one or more anti-viral antisense and other nucleic acid components;
(ii) precipitating the one or more anti-viral antisense and other nucleic acid components with one or more salts of a metal;
(iii) heat treating the one or more anti-viral antisense and other nucleic acid components to provide one or more hydrolyzed and heat inactivated anti-viral antisense and other nucleic acid components; and
(iv) formulating one or more hydrolyzed and heat inactivated anti-viral antisense and other nucleic acid components into the solid oral composition.

Provided in another aspect is a process of producing a solid oral composition according to any one of the compositions described herein, the process comprising:
(i) hydrolyzing one or more anti-viral antisense and other nucleic acid components and one or more SARS-CoV antigens and alloantigens;
(ii) precipitating one or more anti-viral antisense and other nucleic acid components and one or more SARS-CoV antigens and alloantigens with one or more salts of a metal;
(iii) heat treating one or more anti-viral antisense and other nucleic acid components and one or more SARS-CoV antigens and alloantigens to provide one or more hydrolyzed and heat inactivated anti-viral antisense and other nucleic acid components and one or more one or more hydrolyzed and heat inactivated SARS-CoV antigens and alloantigens; and
(iv) formulating one or more hydrolyzed and heat inactivated anti-viral antisense and other nucleic acid components and one or more hydrolyzed and heat inactivated SARS-CoV antigens and alloantigens into the solid oral composition.

In some embodiments, hydrolyzing comprises acid treatment. In some embodiments, precipitation provides for the formation a metal salt matrix, wherein the one or more anti-viral antisense and other nucleic acid components and optionally, the one or more SARS-CoV antigens and alloantigens, are embedded in the metal salt matrix. In some embodiments, the metal salt matrix further comprises one or more immune-modulating agents.

### Methods of Treatment

The compositions describing herein are useful for treating organ and infection site injuries caused by SARS-CoV-2 (COVID-19), Delta and other variants of SARS-CoV-2, SARS-CoV-1 (SARS), MERS, and pandemic influenza viruses. These viruses are known to cause hypercytokinemia (commonly known as cytokine storm), which is characterized by a hyperreactive and uncontrolled immune response and results in the production of high levels of pro-inflammatory cytokines and chemokines. If there high levels of pro-inflammatory cytokines and chemokines are left unchecked or untreated, this may result in long lasting lung and organ injuries, and even death. As shown by the Examples, the compositions described herein can deliver a therapeutic amount of the active agent (e.g., anti-viral antisense and other nucleic acid components, SARS-associated coronavirus (SARS-CoV) antigens and alloantigens, immune-modulating agents, or a combination thereof) directly to the infection sites, where the composition can directly repair the organ damage and the cytokine storm effects by its anti-inflammatory properties.

The compositions described herein are administered to deliver a therapeutically effective amount of the active agents (e.g., anti-viral antisense and other nucleic acid components, SARS-associated coronavirus (SARS-CoV) antigens and alloantigens, immune-modulating agents, or a combination thereof) to stimulate humoral (antibody) immunity, cellular immunity, and mucosal immunity in the subject. In some embodiments, the compositions described herein are administered to deliver a therapeutically effective amount of the one or more hydrolyzed and heat inactivated anti-viral antisense and other nucleic acid components to stimulate humoral (antibody) immunity, cellular immunity, and mucosal immunity in the subject.

Provided in one aspect is a method of preventing or treating coronavirus disease 2019 (COVID-19) and COVID-19 related complications in a subject in need thereof comprising administering a therapeutically effective amount of any one of the compositions described here. In some embodiments, the COVID-19 and COVID-related complications is caused by wildtype or variants of the SARS-CoV-2 virus. In some embodiments, the variant of the SARS-CoV-2 virus is the Delta variant.

Provided in one aspect is a method of treating hypercytokinemia (also, known as cytokine storm) and virus-induced organ damage associated with a virus selected from coronavirus disease 2019 (COVID-19), severe acute respiratory syndrome (SARS), Middle East respiratory syndrome (MERS), and influenza in a subject in need thereof comprising administering a therapeutically effective amount of any one of the compositions described herein.

In any embodiment, the subject in need thereof is a human or an animal.

### Combination Therapy

The compositions described herein may be administered in combination with an anti-tuberculosis drug. The compositions or therapies described herein may be used in combination with any other bioactive substances, which include but are not limited to pharmaceutically effective substances, including, but not limited to, anti-inflammatory drugs, analgesics, tranquillizers, antianxiety drugs, antispasmodics, antidepressants, antipsychotics, antianxiety drugs, narcotic antagonists, anti-parkinsonism agents, cholinergic agonists, chemotherapeutic drugs, immunosuppressive agents, antiviral drugs, anti-microbial drugs like antibiotics, appetite suppressants, anticholinergics, antimetrics, antihistaminics, antimigraine agents, coronary, cerebral or peripheral vasodilators, hormonal agents, contraceptives, antithrombotic agents, diuretics, antihypertensive agents, cardiovascular drugs, opioids, and the like.

The present invention, thus generally described, will be understood more readily by reference to the following examples, which are provided by way of illustration and are not intended to be limiting of the present invention.

### EXAMPLES

### Example 1

The molecular basis of the “key and lock” mechanism of HAP-V+ and HAP-VL for SARS-CoV-2 virus neutralization in the compositions described herein is mediated, in part, by the antisense sequences. These antisense sequences can either suppress or block the gene expression of SARS-CoV-2 viral proteins in host cells, thereby inhibit the viral replication, resulting in therapeutic effectiveness. Furthermore, these antisense molecules are embedded in nanoparticle or magnesium and/or calcium salt matrix that serve as effective delivery and sustained release vaccine systems.

The below table shows the components present in the nanoparticle matrix magnesium composition of the HAP-V+ and HAP-VL therapeutic vaccines. This nanoparticle magnesium matrix embeds the molecular and biological molecules, protecting them from acid, heat inactivation during oral tablet production process, and from enzymatic and acid degradations following oral administration in the host to ensure optimum therapeutic outcomes.

TABLE 1

| Components | Amount (mg) |
|---|---|
| Magnesium chloride hexahydrate | 335 |
| (equivalent to magnesium 40.0 mg) | |
| Lactose Monohydrate | 255 |
| Tapioca Starch | 185 |
| Talc | 10 |
| Magnesium Stearate | 5 |
| Color Film | 10 |

The below table shows the siRNA and primer sequences that allow amplification of antisense molecules to be incorporated into HAP-V+ and HAP-VL for the oral tablet production process, and allow for inhibition of SARS-CoV-2 virus entry and replication in the host. The siRNA in the below table was from selected siRNA for MER-COV (orflab gene) and SAR-COV (ORF1b, Spike).

TABLE 2

| | | | | Seed- |
|---|---|---|---|---|
| | | | | duplex |
| | Location | | | stability |
| | of siRNA | | | (Tm/° C.) |
| | in viral | | Predicted RNA | Guide/ |
| | genome | | oligo sequences | Passenger |
| No. | (Start-End) | Target sequence | 5′-3′ | strand |
| 1 | 791-813 | AGCAATCTATTTTTA | UAAUAGUAAAAAUAGAUUGCU | 6.3/6.6 |
| | | CTATTAAT | CAAUCUAUUUUUACUAUUAAU | |
| | | (SEQ ID NO: 1) | (SEQ ID NO: 2) | |
| 2 | 1615-1637 | ATGGATAATGCTATT | AACAUUAAUAGCAUUAUCCAU | 6.9/8.7 |
| | | AATGTTGG | GGAUAAUGCUAUUAAUGUUGG | |
| | | (SEQ ID NO: 3) | (SEQ ID NO: 4) | |
| 3 | 1910-1932 | GCGACTTTATGTCTA | UAAUUGUAGACAUAAAGUCGC | 6.9/4.6 |
| | | CAATTATT | GACUUUAUGUCUACAAUUAUU | |
| | | (SEQ ID NO: 5) | (SEQ ID NO: 6) | |
| 4 | 4018-4040 | GACACTTTAGATGAT | UAAGAUAUCAUCUAAAGUGUC | 6.6/9.8 |
| | | ATCTTACA | CACUUUAGAUGAUAUCUUACA | |
| | | (SEQ ID NO: 7) | (SEQ ID NO: 8) | |
| 5 | 5597-5619 | ATGCTATTAGTTTGA | UAAAACUCAAACUAAUAGCAU | 13.3/2.8 |
| | | GTTTTAAT | GCUAUUAGUUUGAGUUUUAAU | |
| | | (SEQ ID NO: 9) | SEQ ID NO: 10) | |
| 6 | 5598-5620 | TGCTATTAGTTTGAG | UUAAAACUCAAACUAAUAGCA | 4.9/6.3 |
| | | TTTTAATA | CUAUUAGUUUGAGUUUUAAUA | |
| | | (SEQ ID NO: 11) | (SEQ ID NO: 12) | |
| *7 | 5819-5841 | GAGCTAGTTTGCGTC | AAAUUUGACGCAAACUAGCUC | 7.4/9.8 |
| | | AAATTTTT | GCUAGUUUGCGUCAAAUUUUU | |
| | | (SEQ ID NO: 13) | (SEQ ID NO: 14) | |
| 8 | 9495-9517 | CTCTAATATCTTTGT | UUAAUAACAAAGAUAUUAGAG | 8.1/7.2 |
| | | TATTAACA | CUAAUAUCUUUGUUAUUAACA | |
| | | (SEQ ID NO: 15) | (SEQ ID NO: 16) | |
| 9 | 9533-9555 | CTCTTAGAAACTCTT | UAGUUAAAGAGUUUCUAAGAG | 7.3/6.6 |
| | | TAACTAAT | CUUAGAAACUCUUUAACUAAU | |
| | | (SEQ ID NO: 17) | (SEQ ID NO: 18) | |
| 10 | 13605-13627 | TGGTTTGATTTTGTT | AUUUUCAACAAAAUCAAACCA | 9.5/8.4 |
| | | GAAAATCC | GUUUGAUUUUGUUGAAAAUCC | |
| | | (SEQ ID NO: 19) | (SEQ ID NO: 20) | |
| *11 | 4022-4041 | CGAGGATTAATGTGA | GCUCCUAAUUACACUCAACCG | 7.3/9.6 |
| | | GTTG | AGGAUUAAUGUGAGUUG | |
| | | (SEQ ID NO: 21) | (SEQ ID NO: 22) | |
| *12 | 5012-5031 | CCTACTCCTTCCGTT | GGAUGAGGAAGGCAAUUUACC | 7.2/9.8 |
| | | AAAT | UACUCCUUCCGUUAAAU | |
| | | (SEQ ID NO: 23) | (SEQ ID NO: 24) | |

MER-COV sequence accession number: KF745068 (France), KT036372 (China), KF917527 (Jeddha) SAR-COV sequence accession number: AY350750, NC-004718 and AY274119 (China)
*GC content is 36-52% for both sense and antisense strand.
*Reverse transfection test in Vero cells shows its effective (Test by Western blot and Northern blot analysis)

siRNA Production

The following criteria was used to select the siRNA:
1. siRNAs with 36-52% GC content were chosen as these are more active than those with a higher G/C content.
2. 4-6 nucleotide poly(T) tract acted as a termination signal for RNA pol III. Stretches of >4 T's or A's in the target sequence were avoided when designing sequences that were expressed from an RNA pol III promoter.
3. The potential target sites were compared to the appropriate genome database (human, mouse, rat, etc.) and any target sequences with more than 16-17 contiguous base pairs of homology to other coding sequences were eliminated from consideration.

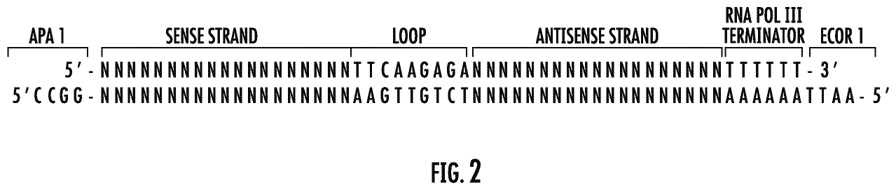

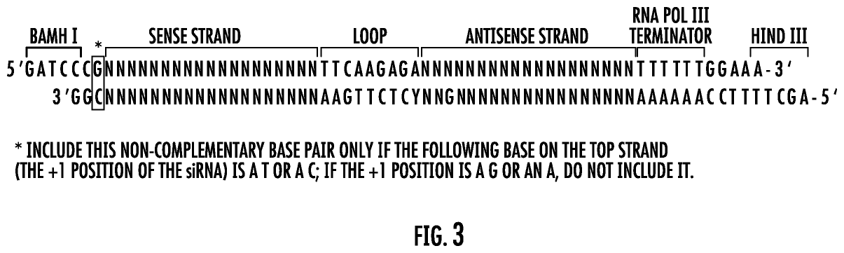

The following steps were used to construct the siRNA library (cloning) into plasmid vector (pSilencer vectors):
1. Target sequence of siRNA with flanking region in viral genome; MERs-COV accession No. KT036372 strain and SAR-COV accession No. AY350750 were amplified by convention PCR using specific primers.
2. In general, the RNA oligonucleotides consisted of a 19-nucleotide sense siRNA sequence linked to its reverse complementary antisense siRNA sequence by a short spacer. The inventors have successfully used a 9-nucleotide spacer (TTCAAGAGA), although other spacers can be designed. 5-6 T's were added to the 3′ end of the oligonucleotide. In addition, for cloning into the pSilencer 1.0-U6 vector, nucleotide overhangs to the EcoR I and Apa I restriction sites were added to the 5′ and 3′ end of the DNA oligonucleotides, respectively (FIG. 2). In contrast, for cloning into the pSilencer 2.0-U6, 2.1-U6, 3.0-H1, or 3.1-H1 vectors, nucleotide overhangs with BamH I and Hind III restriction sites were added to the 5′ and 3′ end of the DNA oligonucleotides, respectively (FIG. 3).

FIG. 2 shows the Insert Design for pSilencer 1.0-U6. This insert is specific for the pSilencer 1.0-U6 Vector and contains the appropriate 3′ overhangs for directional cloning into this vector. The loop sequence and length can be varied as desired.

FIG. 3 shows the Insert Design for pSilencer 2.0-U6 and pSilencer 3.0-H1. The insert design is specific for the pSilencer 2.0-U6, 2.1-U6, 3.0-H1 and 3.1-H1 Expression Vectors and contains the appropriate overhanging 5′ ends for directional cloning into these plasmids. As with p Silencer 1.0-U6 shown in FIG. 2, early indications suggest that a great deal of latitude is possible in the design of the loop. Provided herein is one loop sequence that the inventors found worked well.

The following outlines the procedures for selecting the siRNA by reverse transfection test.

The materials for the reverse transfection test were:
The SAR-COV strain and MER-COV strain for study. The inventors started with PUMC-01 (AY350750) and KT036372, isolated from a patient in the Peking Union Medical Hospital (PUMH) and propagated in cultured Vero cells. A titer of 105 TCID50/1.0 mL was needed.
Vero cells for studies of Coronavirus infection and siRNA effect.
Bio-safety level 3 (BSL-3) facility for the virus.
Synthetic siRNA or siRNA extract
Carriers: D5W (5% glucose in sterile water); Infasurf™ (Forest Labs, St. Louis, MO).
Reagents for viral growth and assay
Standard procedures and reagents for the isolation of nucleic acids and for PCR.

The following steps were used for the reverse transfection test:
Infect Vero cells by Coronavirus, with or without treatment of siSC2-5, harvest and fix in 2.5% glutaraldehyde for 4 h and postfix in 1% osmium tetroxide for 1 h.
Transfer the cells to a 1.5-mL tube and centrifuge at 1,000 rpm for 10 min.
Remove the supernatant and add a liquidized 2% agarose (Sigma, St. Louis, MO) solution at 55-60° C. to the cell pellet. After the gel solidifies, prepare approximately 1-mm3 cubes containing cell pellet and dehydrate in graded ethanol.
Embed the cubes in epoxy resin (Polysciences, Warrington, RI). Prepare ultrathin sections (70 nm thick) and stain with uranyl acetate and lead citrate (Leica Microsystems, Vienna, Austria).
Examine the sections under an electron microscope at 80 kV. Mark the images with a 200-nm-long scale bar.

### HAP-V Production Process

The following describes the steps used to prepare the HAP-V compositions.

Step 1: Prepare selected siRNA (siRNA Production). To increase stability, the inventors incorporated “dTdT” modification to the end 3′ of the antisense siRNAs by plasmid insertion cloning.

Step 2: Prepare cationic cell penetrating protein from HIV-1 and Mg nanoparticle with siRNA. The cell penetrating protein was prepared by obtaining whole blood samples from about 10-20 AIDS patients (about 100-200 cc per patient), that is, patients who were HIV positive. It was not necessary to have an equal amount of blood from each patient. Alternatively, the HIV virus can be cultivated in blood in which case the inoculated blood can then be processed. Currently, a cultivated HIV-1 in T-Cell is being used.

The cultivated HIV-1 T-Cell was placed in a first chamber. Distilled water, selected siRNA plasmid, carbon dioxide gas and magnesium bicarbonate were charged to a separate closed chamber, provided with an agitator, which was cooled. Cold contents from the second chamber were added to a third chamber fitted with an ultrasonic homogenizer. Pooled blood from the first chamber was added to the third chamber. The temperature was raised to about 80-90° C. in the homogenizer chamber. The homogenizer chamber was operated at about 30,000 rpm for about 30-45 minutes. A mechanical, high speed agitator can be used in lieu of an ultrasonic homogenizer. Carbon dioxide gas was evolved which is exhausted to the atmosphere. The liquid aqueous contents in the homogenizer chamber were then allowed to cool and forming a solid precipitate.

The solids in the homogenizer chamber were collected on filter paper, #42 or #45. The filtrate was washed 2-4 times with distilled water. The filtrate was oven dried at about 80° C. for 8 hours. The powdered filtrate may be grounded finer in a grinder. The resulting product was a fine powder having an average particle size of smaller than 10 microns. The fine powder was sterilized at about 120° C. at 15 psig for 1 hour.

### Example 2. HAP-V+ Blocking of SARS-CoV-2 Virus Entry and Replication in Target Host Cells

Antiviral-based Cytopathogenic Effect (CPE) inhibition assay was used to evaluate the ability of HAP-V+ to protect host cells from the cytopathogenic effect of SARS-CoV-2 and Delta variant. Cytopathogenic effect refers to the ability of the virus to induce morphological changes to, or the killing of host cells. To this end, Vero cells monolayer were incubated with various concentrations of the HAP-V+ solution. These cells were then infected with 200 TCID50 dilution of the virus. After incubation for 1, 2, 3 days at 37° C., the CPE was quantitated visually from the infected Vero cells. The results are shown below.

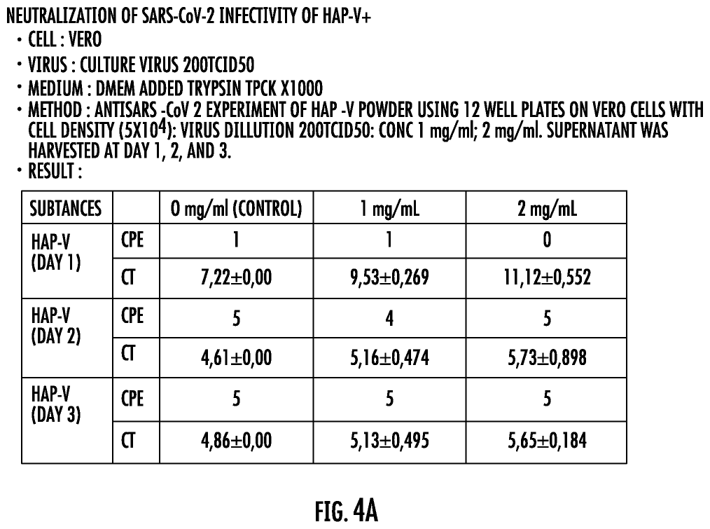

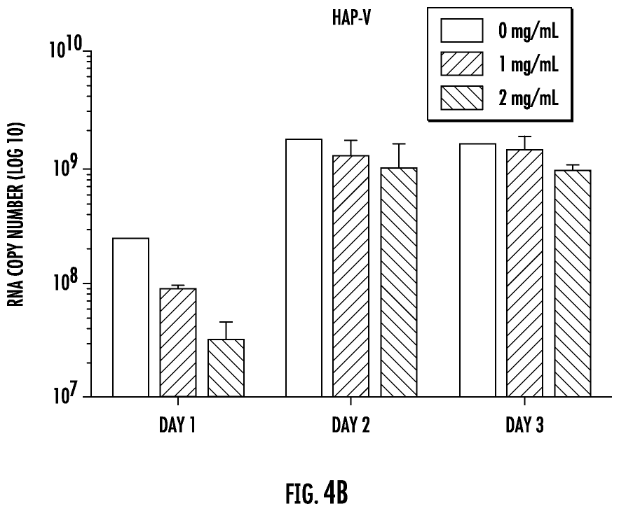

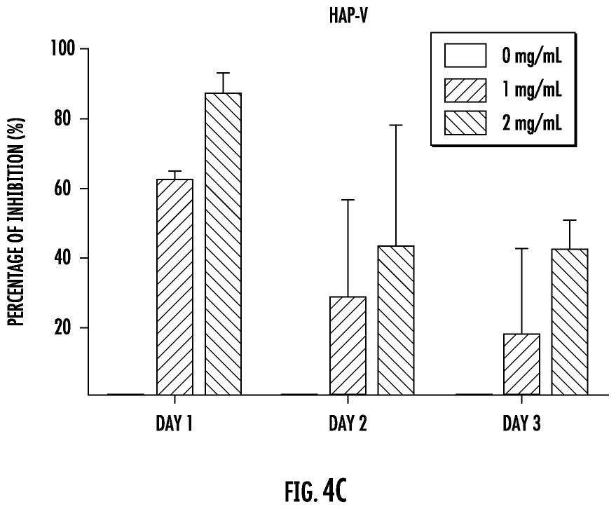

FIGS. 4A, 4B, and 4C show the inhibition of cytopathogenic effects (CPE) and infectivity of SARS-CoV-2 wildtype strain in African green monkey's kidney epithelial cells (Vero cells). FIG. 4A shows the neutralization of SARS-CoV-2 virus by various concentrations of HAP-V+ (1-2 mg/mL) in Vero cells as measured by the cytopathathic effect (CPE) and cycle threshold (CT) at day 1-3 post treatment. FIG. 4B shows the inhibition of viral replication by HAP-V+ in Vero cells by measured by the number of viral RNA copy numbers at day 1-3 post treatment. FIG. 4C shows neutralization of SARS-COV-2 by HAP-V+ as expressed as % viral inhibition.

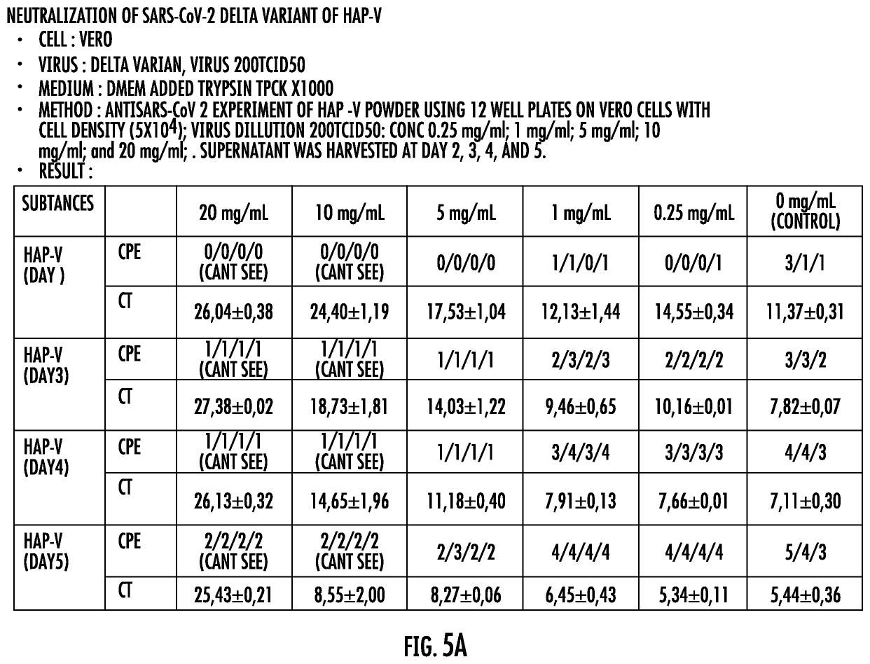

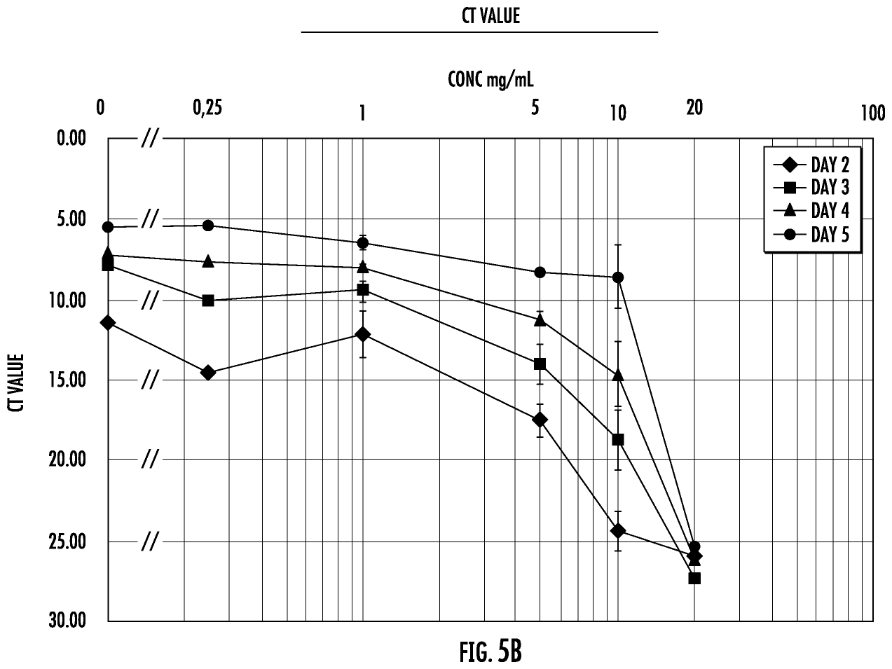

FIGS. 5A and 5B show the inhibition of cytopathogenic effects (CPE) and infectivity of SARS-CoV-2 Delta variant strain in African green monkey's kidney epithelial cells (Vero cells). FIG. 5A shows the neutralization of Delta variant strain of SARS-CoV-2 virus by various concentrations of HAP-V+ (0.25 to 20 mg/mL) in Vero cells as measured by the cytopathathic effect (CPE) and cycle threshold (CT) at day 2-5 post treatment FIG. 5B shows inhibition of viral infectivity of the Delta variant strain of SARS-CoV-2 as measured using the CPE assay and expressed as cycle threshold (CT) reduction values.

### Example 3. Treatment of Cytokine Storm-Related Lung Injury and Inflammation in COVID Patient Suffering from Long Term Clinical Effects of COVID-19 Infection

A male person suffering from long term severe effects of SARS-CoV-2 infection presented himself at a local university hospital in Thailand. The patient was recovering from COVID but his symptoms including heavy and difficult breathing problem persisted. A follow-up chest radiograph revealed multiple foci lung pathological features which include ground glass opacity (cloud formation) and/or consolidations in the bilateral lungs. These abnormal pathological features in the lungs were considered to the most likely causes of the patient's respiratory, breathing problems.

Upon careful consideration, his attending physician recommended treatment with HAP-VL, which is a magnesium-based therapeutic vaccine developed to treat COVID-associated, cytokine storm-related, organ and lung injuries. The HAP-VL was prepared in accordance to the procedure described in Example 1 and contains the anti-viral antisense or siRNA components that target the viral mRNA that encodes for the various the viral nucleocapsid, spike glycoprotein, membrane and envelop proteins and RNA polymerase, inactivated SARS-CoV antigens and alloantigens and an immune-modulating agents that include anti-inflammatory or lung or brain tissue healing agents. Upon daily dosing with HAP-VL, the patient's breathing started to improve, and within two weeks after treatment, the patient felt almost completely free of respiratory problems. A chest radiograph was taken of the patient after one month of treatment. The HAP-VL post treatment chest radiograph reveals significant reduced ground glass opacity in both lungs and reduced consolidations in the lungs. Some residual opacity and consolidation remain but these are expected to improve and recover upon continual dosing of HAP-VL.

Para. 1. A composition comprising one or more hydrolyzed and heat inactivated anti-viral antisense and other nucleic acid components, wherein the composition is administered to a subject in need thereof.

Para. 2. The composition of Para. 1, wherein the one or more hydrolyzed and heat inactivated anti-viral antisense and other nucleic acid components comprise one or more genomic RNA sequences, immunomodulating CpG motifs, and small interfering RNAs (siRNAs).

Para. 3. The composition of Paras. 1 or 2, wherein the one or more hydrolyzed and heat inactivated anti-viral antisense and other nucleic acid components comprises one or more small interfering RNA (siRNA) targeting expression of SARS-CoV-2 viral proteins.

Para. 4. The composition of any one of Paras. 1-3, wherein the one or more hydrolyzed and heat inactivated anti-viral antisense and other nucleic acid components comprises one or more small interfering RNA (siRNA) comprising an oligonucleotide sequence as set forth in any one of SEQ ID NOS: 2, 4, 6, 8, 10, 12, 14, 16, 18, 20, 22, and 24.

Para. 5. The composition of any one of Paras. 1-3, wherein the one or more hydrolyzed and heat inactivated anti-viral antisense and other nucleic acid components comprises one or more small interfering RNA (siRNA) comprising an oligonucleotide sequence as set forth in SEQ ID NO: 14, SEQ ID NO: 22, or SEQ ID NO: 24.

Para. 6. The composition of any one of the preceding Paras., further comprising one or more hydrolyzed and heat-inactivated SARS-associated coronavirus (SARS-CoV) antigens and alloantigens derived from pooled blood or serum of a species that is the same as the subject.

Para. 7. The composition of Para. 6, wherein the one or more hydrolyzed and heat-inactivated SARS-CoV antigens and alloantigens comprise one or more selected from viral proteins, nucleocapsids, and genomic RNA sequences.

Para. 8. The composition of Para. 7, wherein the one or more viral proteins comprise one or more glycoproteins, envelope and membrane proteins, nucleoproteins, and hemagglutinin-esterase proteins.

Para. 9. The composition of of any one of the preceding Paras., further comprising one or more hydrolyzed and heat-inactivated immune-modulating agents.

Para. 10. The composition of Para. 9, wherein the one or more one hydrolyzed and heat-inactivated immune-modulating agents having anti-inflammatory and antiviral activities.

Para. 11. The composition of Paras. 9 or 10, wherein the one or more one hydrolyzed and heat-inactivated immune-modulating agents comprise one or more immune-modulating cytokines, adjuvants, CpG motifs, peptides, RNA and DNA sequences, and anti-inflammatory molecules.

Para. 12. The composition of any one of the preceding Paras., wherein the one or more hydrolyzed and heat inactivated anti-viral antisense and other nucleic acid components, one or more hydrolyzed and heat-inactivated SARS-associated coronavirus (SARS-CoV) antigens and alloantigens, one or more one hydrolyzed and heat-inactivated immune-modulating agents, or a combination thereof are each independently embedded in a matrix comprising one or more salts of a metal.

Para. 13. The composition of Para. 12, wherein the metal is selected from a group of alkali metals, alkaline earth metals, transition metals, metalloids, basic metals, and rare earth elements.

Para. 14. The composition of Para. 12, wherein the metal is magnesium, calcium, lithium, beryllium, sodium, silicon, magnesium, aluminium, aluminum, potassium, titanium, vanadium, chromium, manganese, cobalt, nickel, copper, zinc, zirconium, molybdenum, silver, arsenic, cadmium, antimony, barium, osmium, platinum, or gold.

Para. 15. The composition of Para. 12, wherein the metal is magnesium or calcium.

Para. 16. The composition of Para. 12, wherein the one or more salts of the metal comprise boride, acetate, phosphate, aluminide, aspartate, benzoate, bromide, carbonate, chloride, chloride hexahydrate, citrate, diboride, diglutamate, diuranate, fluoride, gluconate, hexahydrate, hydride, hydroxide, oxide, iodide, lactate, levulinate, nitrate, nitride, orotate, oxychloride, oxysulfate, perchlorate, peroxide, pidolate, silicide, stearate, sulfate, sulfide, sulfite, and trisilicate salt.

Para. 17. The composition of any one of the preceding Paras., wherein the composition is formulated for oral administration, intranasal administration, intramuscular injection, aerosol delivery or intravenous injection.

Para. 18. The composition of Para. 17, wherein the composition is formulated for oral administration.

Para. 19. The composition of Para. 18, wherein the composition is formulated as a pill or tablet.

Para. 20. The composition of Para. 19, wherein the composition further comprises one or more of excipients selected from a binder, starch or lactose, a disintegrating agent, a lubricant, a glidant, a sweetening agent, and a flavoring agent.

Para. 21. The composition of Para. 20, where composition further comprises lactose monohydrate, tapioca starch, talc, and magnesium stearate.

Para. 22. The composition of any one of the preceding Paras., wherein the composition is room temperature stable.

Para. 23. The composition of Para. 22, wherein the composition is room temperature stable for at least one year, two years, three years, four years, or five years.

Para. 24. The composition of any one of the preceding Paras., wherein the composition does not require storage at low temperatures and/or refrigeration.

Para. 25. The composition of any one of the Paras., wherein the composition is administered to deliver a therapeutically effective amount of the one or more hydrolyzed and heat inactivated anti-viral antisense and other nucleic acid components to stimulate humoral (antibody), cellular, and mucosal immunity in the subject.

Para. 26. A method of preventing or treating coronavirus disease 2019 (COVID-19) and COVID-19 related complications in a subject in need thereof comprising administering a therapeutically effective amount of a composition of any one of Paras. 1-25.

Para. 27. The method of Para. 26, wherein the COVID-19 and COVID-related complications is caused by wildtype or variants of the SARS-CoV-2 virus.

Para. 28. The method of Para. 27, wherein the variant of the SARS-CoV-2 virus is the Delta variant.

Para. 29. A method of treating hypercytokinemia (commonly called cytokine storm) and virus-induced organ damage associated with a virus selected from coronavirus disease 2019 (COVID-19), severe acute respiratory syndrome (SARS), Middle East respiratory syndrome (MERS), and influenza in a subject in need thereof comprising administering a therapeutically effective amount of a composition of any one of Paras. 1-25.

Para. 30. The method of any one of the Paras. 26-29, wherein the subject in need thereof is a human or an animal.

Para. 31. A process of producing a solid oral composition according to a composition of any one of Paras. 1-25, the process comprising:
(i) hydrolyzing one or more anti-viral antisense and other nucleic acid components;
(ii) precipitating the one or more anti-viral antisense and other nucleic acid components with one or more salts of a metal;
(iii) heat treating the one or more anti-viral antisense and other nucleic acid components to provide one or more hydrolyzed and heat inactivated anti-viral antisense and other nucleic acid components; and
(iv) formulating one or more hydrolyzed and heat inactivated anti-viral antisense and other nucleic acid components into the solid oral composition.

Para. 32. A process of producing a solid oral composition according to a composition of any one of Paras. 6-25, the process comprising:
(i) hydrolyzing one or more anti-viral antisense and other nucleic acid components and one or more SARS-CoV antigens and alloantigens;
(ii) precipitating one or more anti-viral antisense and other nucleic acid components and one or more SARS-CoV antigens and alloantigens with one or more salts of a metal;
(iii) heat treating one or more anti-viral antisense and other nucleic acid components and one or more SARS-CoV antigens and alloantigens to provide one or more hydrolyzed and heat inactivated anti-viral antisense and other nucleic acid components and one or more one or more hydrolyzed and heat inactivated SARS-CoV antigens and alloantigens; and
(iv) formulating one or more hydrolyzed and heat inactivated anti-viral antisense and other nucleic acid components and one or more hydrolyzed and heat inactivated SARS-CoV antigens and alloantigens into the solid oral composition.

Para. 33. The process of Paras. 31 or 32, wherein hydrolyzing comprises acid treatment.

Para. 34. The process of any one of Paras. 31-33, wherein precipitation provides for the formation a metal salt matrix, wherein the one or more anti-viral antisense and other nucleic acid components and optionally, the one or more SARS-associated coronavirus (SARS-CoV) antigens and alloantigens, are embedded in the metal salt matrix.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

1. A composition comprising one or more hydrolyzed and heat inactivated anti-viral antisense and other nucleic acid components, wherein the composition is administered to a subject in need thereof.

2. The composition of claim 1, wherein the one or more hydrolyzed and heat inactivated anti-viral antisense and other nucleic acid components comprise one or more genomic RNA sequences, immunomodulating CpG motifs, and small interfering RNAs (siRNAs).

3. The composition of claim 1, wherein the one or more hydrolyzed and heat inactivated anti-viral antisense and other nucleic acid components comprises one or more small interfering RNA (siRNA) targeting expression of SARS-CoV-2 viral proteins.

4. The composition of claim 1, wherein the one or more hydrolyzed and heat inactivated anti-viral antisense and other nucleic acid components comprises one or more small interfering RNA (siRNA) comprising an oligonucleotide sequence as set forth in any one of SEQ ID NOS: 2, 4, 6, 8, 10, 12, 14, 16, 18, 20, 22, and 24.

5. (canceled)

6. The composition of claim 1, further comprising one or more hydrolyzed and heat-inactivated SARS-associated coronavirus (SARS-CoV) antigens and alloantigens derived from pooled blood or serum of a species that is the same as the subject.

7. The composition of claim 6, wherein the one or more hydrolyzed and heat-inactivated SARS-CoV antigens and alloantigens comprise one or more selected from viral proteins, nucleocapsids, and genomic RNA sequences, and optionally, wherein the one or more viral proteins comprise one or more glycoproteins, envelope and membrane proteins, nucleoproteins, and hemagglutinin-esterase proteins.

8. (canceled)

9. The composition of claim 1, further comprising one or more hydrolyzed and heat-inactivated immune-modulating agents.

10. The composition of claim 9, wherein the one or more one hydrolyzed and heat-inactivated immune-modulating agents having anti-inflammatory and antiviral activities and/or wherein the one or more one hydrolyzed and heat-inactivated immune-modulating agents comprise one or more immune-modulating cytokines, adjuvants, CpG motifs, peptides, RNA and DNA sequences, and anti-inflammatory molecules.

11. (canceled)

12. The composition of claim 1, wherein the one or more hydrolyzed and heat inactivated anti-viral antisense and other nucleic acid components, one or more hydrolyzed and heat-inactivated SARS-associated coronavirus (SARS-CoV) antigens and alloantigens, one or more one hydrolyzed and heat-inactivated immune-modulating agents, or a combination thereof are each independently embedded in a matrix comprising one or more salts of a metal.

13. The composition of claim 12, wherein the metal is selected from a group of alkali metals, alkaline earth metals, transition metals, metalloids, basic metals, and rare earth elements and/or wherein the metal is magnesium, calcium, lithium, beryllium, sodium, silicon, magnesium, aluminium, aluminum, potassium, titanium, vanadium, chromium, manganese, cobalt, nickel, copper, zinc, zirconium, molybdenum, silver, arsenic, cadmium, antimony, barium, osmium, platinum, or gold.

14. (canceled)

15. (canceled)

16. The composition of claim 12, wherein the one or more salts of the metal comprise boride, acetate, phosphate, aluminide, aspartate, benzoate, bromide, carbonate, chloride, chloride hexahydrate, citrate, diboride, diglutamate, diuranate, fluoride, gluconate, hexahydrate, hydride, hydroxide, oxide, iodide, lactate, levulinate, nitrate, nitride, orotate, oxychloride, oxysulfate, perchlorate, peroxide, pidolate, silicide, stearate, sulfate, sulfide, sulfite, and trisilicate salt.

17. The composition of claim 1, wherein the composition is formulated for oral administration, intranasal administration, intramuscular injection, aerosol delivery or intravenous injection.

18. (canceled)

19. (canceled)

20. The composition of claim 17, wherein the composition further comprises one or more of excipients selected from a binder, starch or lactose, a disintegrating agent, a lubricant, a glidant, a sweetening agent, and a flavoring agent.

21. (canceled)

22. The composition of claim 1, wherein the composition is room temperature stable and/or wherein the composition is room temperature stable for at least one year, two years, three years, four years, or five years.

23. (canceled)

24. The composition of claim 1, wherein the composition does not require storage at low temperatures and/or refrigeration.

25. The composition of claim 1, wherein the composition is administered to deliver a therapeutically effective amount of the one or more hydrolyzed and heat inactivated anti-viral antisense and other nucleic acid components to stimulate humoral (antibody), cellular, and mucosal immunity in the subject.

26. A method of preventing or treating coronavirus disease 2019 (COVID-19) and COVID-19 related complications in a subject in need thereof comprising administering a therapeutically effective amount of a composition of claim 1.

27. The method of claim 26, wherein the COVID-19 and COVID-related complications is caused by wildtype or variants of the SARS-CoV-2 virus, and optionally, wherein the variant of the SARS-CoV-2 virus is the Delta variant.

28. (canceled)

29. A method of treating hypercytokinemia (commonly called cytokine storm) and virus-induced organ damage associated with a virus selected from coronavirus disease 2019 (COVID-19), severe acute respiratory syndrome (SARS), Middle East respiratory syndrome (MERS), and influenza in a subject in need thereof comprising administering a therapeutically effective amount of a composition of claim 1.

30. (canceled)

31. A process of producing a solid oral composition according to a composition of claim 1, the process comprising:
(i) hydrolyzing one or more anti-viral antisense and other nucleic acid components;
(ii) precipitating the one or more anti-viral antisense and other nucleic acid components with one or more salts of a metal;
(iii) heat treating the one or more anti-viral antisense and other nucleic acid components to provide one or more hydrolyzed and heat inactivated anti-viral antisense and other nucleic acid components; and
(iv) formulating one or more hydrolyzed and heat inactivated anti-viral antisense and other nucleic acid components into the solid oral composition.

32. A process of producing a solid oral composition according to a composition of claim 6, the process comprising:
(i) hydrolyzing one or more anti-viral antisense and other nucleic acid components and one or more SARS-CoV antigens and alloantigens;
(ii) precipitating one or more anti-viral antisense and other nucleic acid components and one or more SARS-CoV antigens and alloantigens with one or more salts of a metal;
(iii) heat treating one or more anti-viral antisense and other nucleic acid components and one or more SARS-CoV antigens and alloantigens to provide one or more hydrolyzed and heat inactivated anti-viral antisense and other nucleic acid components and one or more one or more hydrolyzed and heat inactivated SARS-CoV antigens and alloantigens; and
(iv) formulating one or more hydrolyzed and heat inactivated anti-viral antisense and other nucleic acid components and one or more hydrolyzed and heat inactivated SARS-CoV antigens and alloantigens into the solid oral composition.

33. The process of claim 31, wherein hydrolyzing comprises acid treatment and/or wherein precipitation provides for the formation a metal salt matrix, wherein the one or more anti-viral antisense and other nucleic acid components and optionally, the one or more SARS-associated coronavirus (SARS-CoV) antigens and alloantigens, are embedded in the metal salt matrix.

34. (canceled)


SEQUENCE LISTING (24 sequences)

SEQ ID NO: 1 - RNA, 23 nt, synthetic construct
  source 1..23: mol_type = other RNA
agcaatctatttttactattaat

SEQ ID NO: 2 - RNA, 42 nt, synthetic construct
  source 1..42: mol_type = other RNA
taatagtaaaaatagattgctcaatctatttttactattaat

SEQ ID NO: 3 - RNA, 23 nt, synthetic construct
  source 1..23: mol_type = other RNA
atggataatgctattaatgttgg

SEQ ID NO: 4 - RNA, 42 nt, synthetic construct
  source 1..42: mol_type = other RNA
aacattaatagcattatccatggataatgctattaatgttgg

SEQ ID NO: 5 - RNA, 23 nt, synthetic construct
  source 1..23: mol_type = other RNA
gcgactttatgtctacaattatt

SEQ ID NO: 6 - RNA, 42 nt, synthetic construct
  source 1..42: mol_type = other RNA
taattgtagacataaagtcgcgactttatgtctacaattatt

SEQ ID NO: 7 - RNA, 23 nt, synthetic construct
  source 1..23: mol_type = other RNA
gacactttagatgatatcttaca

SEQ ID NO: 8 - RNA, 42 nt, synthetic construct
  source 1..42: mol_type = other RNA
taagatatcatctaaagtgtccactttagatgatatcttaca

SEQ ID NO: 9 - RNA, 23 nt, synthetic construct
  source 1..23: mol_type = other RNA
atgctattagtttgagttttaat

SEQ ID NO: 10 - RNA, 42 nt, synthetic construct
  source 1..42: mol_type = other RNA
taaaactcaaactaatagcatgctattagtttgagttttaat

SEQ ID NO: 11 - RNA, 23 nt, synthetic construct
  source 1..23: mol_type = other RNA
tgctattagtttgagttttaata

SEQ ID NO: 12 - RNA, 42 nt, synthetic construct
  source 1..42: mol_type = other RNA
ttaaaactcaaactaatagcactattagtttgagttttaata

SEQ ID NO: 13 - RNA, 23 nt, synthetic construct
  source 1..23: mol_type = other RNA
gagctagtttgcgtcaaattttt

SEQ ID NO: 14 - RNA, 42 nt, synthetic construct
  source 1..42: mol_type = other RNA
aaatttgacgcaaactagctcgctagtttgcgtcaaattttt

SEQ ID NO: 15 - RNA, 23 nt, synthetic construct
  source 1..23: mol_type = other RNA
ctctaatatctttgttattaaca

SEQ ID NO: 16 - RNA, 42 nt, synthetic construct
  source 1..42: mol_type = other RNA
ttaataacaaagatattagagctaatatctttgttattaaca

SEQ ID NO: 17 - RNA, 23 nt, synthetic construct
  source 1..23: mol_type = other RNA
ctcttagaaactctttaactaat

SEQ ID NO: 18 - RNA, 42 nt, synthetic construct
  source 1..42: mol_type = other RNA
tagttaaagagtttctaagagcttagaaactctttaactaat

SEQ ID NO: 19 - RNA, 23 nt, synthetic construct
  source 1..23: mol_type = other RNA
tggtttgattttgttgaaaatcc

SEQ ID NO: 20 - RNA, 42 nt, synthetic construct
  source 1..42: mol_type = other RNA
attttcaacaaaatcaaaccagtttgattttgttgaaaatcc

SEQ ID NO: 21 - RNA, 19 nt, synthetic construct
  source 1..19: mol_type = other RNA
cgaggattaatgtgagttg

SEQ ID NO: 22 - RNA, 38 nt, synthetic construct
  source 1..38: mol_type = other RNA
gctcctaattacactcaaccgaggattaatgtgagttg

SEQ ID NO: 23 - RNA, 19 nt, synthetic construct
  source 1..19: mol_type = other RNA
cctactccttccgttaaat

SEQ ID NO: 24 - RNA, 38 nt, synthetic construct
  source 1..38: mol_type = other RNA
ggatgaggaaggcaatttacctactccttccgttaaat